(19) United States
(12) Patent Application Publication
(10) Pub. No.: US 2006/0241533 A1
(43) Pub. Date: Oct. 26, 2006

(54) Apparatus and method for treatment of damaged tissue

(76) Inventor: Benjamin Geller, (US)

Correspondence Address: WOLF, BLOCK, SCHORR & SOLIS-COHEN LLP, 250 PARK AVENUE, NEW YORK, NY, 10177, US

(21) Appl. No.: 10/553,933

(22) PCT Filed: Apr. 22, 2004
(86) PCT No.: PCT/IL04/00344

§ 371 Date: Oct. 20, 2005

(30) Foreign Application Priority Data
Apr. 22, 2003 (IL) 155546

(51) Int. Cl. A61H 1/00

(52) U.S. Cl. 601/4

(57) ABSTRACT

Apparatus for the ultrasonic treatment of tissue, including: a housing having a space therewithin and an opening adapted for placement against the tissue, the housing being adapted for introducing liquid therein such that when so placed, the space is filled with liquid; and an ultrasonic power source that introduces ultrasonic vibrations toward the damaged tissue, said vibrations having a frequency and power level sufficient to produce cavitation of the liquid at or near the surface of the tissue.

### FIELD OF THE INVENTION

The present invention relates to the use of ultrasonic vibrations for skin treatments, for example to cleaning or treatment of damaged tissue.

### BACKGROUND OF THE INVENTION

It is known in the art to utilize ultrasonic vibrations for the cleaning of various objects, such as machine parts, ball bearings, and surgical instruments. The objects to be cleaned are placed in a liquid, e.g., a detergent solution or a solvent, into which ultrasonic waves are introduced. The ultrasonic waves cause vibrations which affect cleaning of the objects.

It is also known in the medical field to employ ultrasonic vibrations to remove dead tissue and debris from the skin of a person. U.S. Pat. No. 5,656,015, the disclosure of which is incorporated by reference, is concerned with an ultrasonic system comprising a plurality of piezoelectric transducers which are applied to an area of skin to be treated. The affected area can thus be excited from different aspects, so that the ultrasonic waves cause excitation of the skin tissue.

WO 00/10164, the disclosure of which is incorporated by reference, discloses a device for the removal of dead tissue from the skin of a person, wherein an ultrasonic probe is introduced into a housing containing a liquid debriding agent. Ultrasonic vibrations spread throughout the liquid and act on the debriding agent to affect cleaning of the skin.

While prior art devices employ the principles of ultrasonics, which may be utilized for cleaning the skin, there is a need for more thorough cleaning than can be achieved by the ultrasonic vibrations alone or when utilized in conjunction with a debriding agent.

High ultrasonic energy is associated with a phenomenon called cavitation, whereby the vibrations from an ultrasonic transducer cause large numbers of micro-bubbles to implode with great pressure on the surfaces of objects. This triggers a mechanical action that removes unwanted debris from the objects down to a molecular level. The principle of cavitation has been successfully utilized in industry to remove film or dirt from various objects, even from normally inaccessible holes, cracks, and comers. For example, radioactive scale is thus removed from nuclear reactor fuel and control rods. This principle has not, however, been utilized in the medical field in the cleaning of damaged tissue. In general, such systems are much larger than a wavelength of the acoustic energy.

### SUMMARY OF THE INVENTION

An aspect of the present invention is concerned with the inducement of ultrasonic cavitation on the surface of a tissue, such as human skin.

It is noted that when ultrasonic energy of sufficient energy is injected into a liquid volume, cavitation can occur in the liquid. In general, where the liquid has a free surface (i.e., one that is open to the air), cavitation does not occur at the free surface and within a quarter wave length of the free surface. If the volume has no free surfaces, then cavitation occurs within the bulk of the volume and at surfaces of the enclosure of the liquid.

In an embodiment of the invention, the tissue to be treated is covered by a housing, to form a cavity, into which a liquid is introduced. When an opening of the housing is in contact with the skin it is filled with liquid so that there are no free surfaces of the liquid. A transducer injects ultrasonic energy into the liquid in an amount that is sufficient to induce cavitation at the surface of the tissue. Under these circumstances, the amount of cavitation that takes place near the skin is increased, for the same power, or lower power can be used for the same amount of cavitation.

Optionally, an interface is provided at the ultrasonic power input to the volume of liquid in the housing. The interface preferably has acoustic properties that are similar to those of the liquid. If the transducer were used to inject the acoustic energy directly into the liquid, the interface between the transducer and the liquid would act as a focus for cavitation, resulting in a marked reduction in the acoustic energy that reaches the skin surface, reducing or eliminating cavitation there. This allows for a higher percentage of generated ultrasound energy to be inserted into the liquid, so that desired cavitation at the tissue surface is generated.

In some embodiments of the invention, the cavity is made small enough so that the fields are quasi-static, that is the fields are substantially the same everywhere in the cavity, or at least between the input and the skin.

In an embodiment of the invention, the opening of the housing that touches the skin is formed with a sealing edge. Optionally, the sealing edge is formed of an elastic material. Optionally, the elastic material is formed into a deformable element that securely seals the housing.

In an embodiment of the invention, at least a part of the walls of the cavity are elastic. This allows for changing the shape of the cavity so that the input of the ultrasound can be brought closer to and, in some cases, made to touch the skin. In addition, the soft edge is less likely to irritate or damage the wound.

In an embodiment of the invention, an antibiotic, analgesic, anti-inflammatory or antiseptic material is added to the liquid. The cavitation of the skin, as it removes debris and dead tissue from the surface of the tissue, also exposes live tissue, such that absorption of the additive may be improved. Alternatively, a detergent or saline liquid is used for the cleaning process and removal of debris and therapeutic liquid replaces the cleaning liquid for therapy of the wound. During the therapy, there may or may not be cavitation and the liquid may or may not be flowing.

In an embodiment of the invention, the housing is provided with an inlet for liquid and an outlet for liquid. During operation, as debris is removed, liquid in the housing is replaced, optionally continuously, with the removed debris. Placement of the inlet and outlet induces the removal of the debris from the liquid in the housing. Optionally, the inlet and outlet are placed on opposite sides of the area of the skin being treated, so that the liquid flows across the area, facilitating debris removal from the site of the wound.

In an embodiment of the invention, the housing as a whole is disposable. The transducer and any associated feed in mechanism is reused, but does not have to be sterilized.

There is thus provided, in accordance with an embodiment of the invention, apparatus for the ultrasonic treatment of tissue, including:

a housing having a space therewithin and an opening adapted for placement against the tissue, the housing being adapted for introducing liquid therein such that when so placed, the space is filled with liquid; and

an ultrasonic power source that introduces ultrasonic vibrations toward the damaged tissue, said vibrations having a frequency and power level sufficient to produce cavitation of the liquid at or near the surface of the tissue.

In an embodiment of the invention, the opening comprises a sealing element that provides a seal at the tissue. Optionally, the seal includes a flexible element. Optionally, the seal includes an outwardly protruding portion that is placed to contact the tissue surface. Optionally, the seal includes an inwardly protruding portion that is placed to contact the tissue surface.

In an embodiment of the invention, the ultrasonic power source includes a piezoelectric transducer.

Optionally, the ultrasonic power source generates acoustic energy with a frequency of vibration of not more than 80 kHz. Optionally, the ultrasonic power source generates acoustic energy with a frequency of vibration of not less than 30 kHz.

In an embodiment of the invention, the housing comprises a liquid inlet adapted for introduction of liquid into the space. In an embodiment of the invention, the housing comprises a liquid outlet adapted for the removal of liquid from the space. Optionally, the outlet is disposed at the apex of the space, such that any air in the space is removed via the outlet, when liquid is introduced to fill the space. Optionally, the inlet and the outlet are situated on opposite sides of the opening such that liquid passing through the space from the inlet to the outlet irrigates the tissue.

In an embodiment of the invention, the ultrasonic vibrations are introduced into the space through an acoustic port in the housing. Optionally, the acoustic port has acoustic properties similar to that of said liquid. Optionally, the acoustic port is formed in the housing. Optionally, at least the acoustic port is formed of an elastomer material. Optionally, the distance between the portal and the opening is less than one-half wavelength of the ultrasonic vibrations in the liquid.

Optionally, the housing is formed of an elastomer material. Optionally, at least a portion of the housing is transparent.

In an embodiment of the invention, the housing is formed with a protrusion into the space over a central portion of the opening. Optionally, ultrasonic energy enters said space at said protrusion.

In an embodiment of the invention, the opening is in the form of a right angle cut in the housing, such that the housing is upright when the opening is placed on a horizontal portion of skin.

In an embodiment of the invention, the opening is in the form of an acute angle cut in the housing, such that the housing is upright when the opening is placed on a skin surface that is at an angle to the horizontal.

There is further provided, in accordance with an embodiment of the invention, a method for treating tissue, including:

providing a liquid in contact with a surface of the tissue; and

causing ultrasonic vibrations in the liquid to an extent that cavitation is caused at least at or near the surface of the tissue.

Optionally, the frequency of the ultrasonic vibrations is not more than 80 kHz.

Optionally, not more than 30 kHz.

In an embodiment of the invention, the method includes producing a fluid current moving through the liquid, the current allowing for the removal of debris from the tissue surface.

There is further provided, in accordance with an embodiment of the invention, a method of applying ultrasound to a surface of a patient, comprising:

providing a housing having an opening at one portion thereof and having a source of acoustic energy at a portion of an inner surface thereof;

placing the opening at the patient surface, to form a substantially closed volume in the housing;

filling the volume with liquid, so that all air is removed therefrom; and

activating the source of acoustic energy.

In an embodiment of the invention, activation of the source causes the source to produce sufficient energy to cause cavitation at the patient surface.

### BRIEF DESCRIPTION OF THE DRAWINGS

The present invention is described with respect to the following description of non-limiting exemplary embodiments of the invention, which should be read in conjunction with the following figures. Similar or identical features, which appear in more than one figure are referenced with a same or similar reference numerals. The figures are schematic and the dimensions are chosen for ease of understanding and may not represent actual dimension in an actual device.

FIG. 1 is a schematic illustration of an ultrasonic system for the treatment of damaged slin according to an exemplary embodiment of the invention;

FIG. 2 is a cross-sectional view of an ultrasonic device for the treatment of damaged tissue according to a first embodiment of the invention;

FIG. 3 is a cross-sectional view of an ultrasonic device for the treatment of damaged tissue according to a second embodiment of the invention;

FIG. 4 is a cross-sectional view of the device according to the first embodiment of the invention, wherein manual pressure has been applied to the housing;

FIG. 5 is a cross-sectional view of an ultrasonic device for treatment of damaged skin tissue according to another embodiment of the invention; and

FIG. 6 is a cross-sectional view of an ultrasonic device for treatment of damaged skin tissue according to yet another embodiment of the invention.

### DETAILED DESCRIPTION OF EXEMPLARY EMBODIMENTS

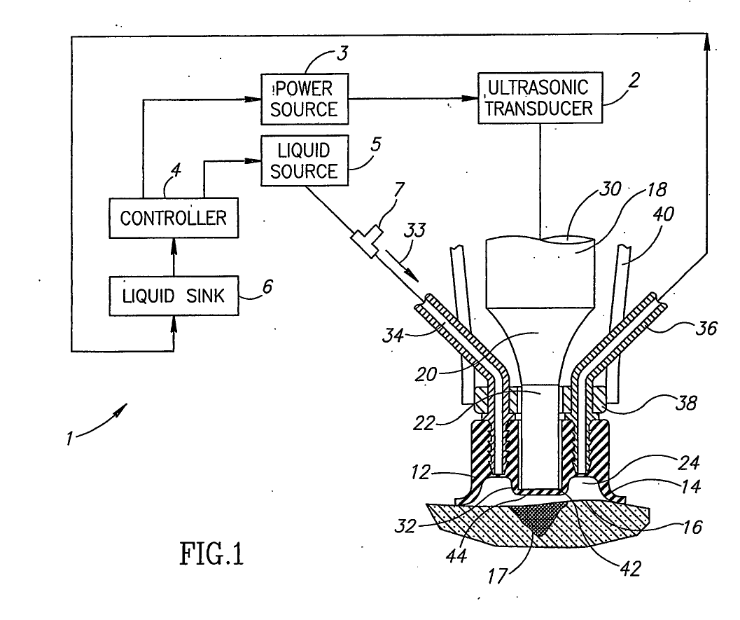

FIG. 1 illustrates an ultrasonic system 1, for treatment of tissue, in accordance with an exemplary embodiment of the invention. System 1 comprises an ultrasonic treatment device 10 (or 50), described in more detail below, a transducer 2, a source of power 3, a controller 4, a source of liquid 5 and a sink for liquid 6. Optionally, an input 7 for a therapeutic agent is also provided.

In operation, according to an exemplary embodiment of the invention, device 10 is placed on a skin surface 16 and liquid is supplied to device 10 until all air is removed from a cavity therein, formed between the device and the skin, as described below. Then, the transducer is activated preferably to cause cavitation in the liquid.

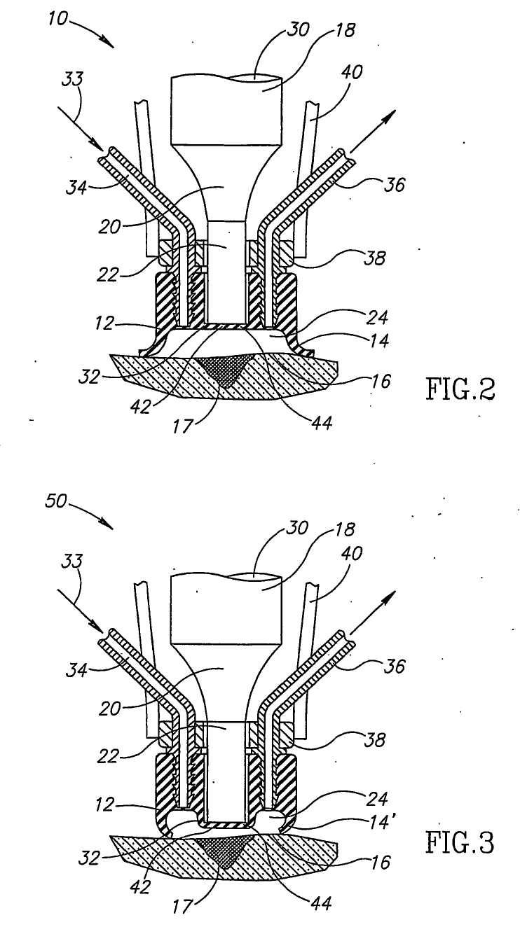

FIGS. 2 and 3 illustrate, in cross-section, ultrasonic devices 10 and 50 for the treatment of damaged tissue, according to embodiments of the present invention. Devices 10 and 50 include an optionally annular housing 12 having a sealing edge, such as the outwardly protruding edge 14 shown in FIG. 1, or an inwardly facing edge 14′, designed so as to be placed on the surface 16 of a tissue, such as a wound 17 in the skin. The sealing edge may be fabricated from any suitable material, and is preferably fabricated from an elastic polymer. Similarly, portions or all of housing 12 may also be made of an elastomer material. Optionally, all or parts of housing 12 may be made of a transparent material, so as to enable the observation of the removal of air bubbles and debris from the wound surface 17, as will be discussed below.

Housing 12 is configured so as to accommodate an optional ultrasound concentrator 30 which transfers and concentrates energy produced by ultrasonic transducer 2 to the wound. Concentrator 30 may be attached to housing 12 in any suitable manner, such as by clamping, and is optionally removable. Optionally, concentrator 30 includes a first cylindrical portion 18 connected to the ultrasonic source, a tapered concentrating portion 20, and a second cylindrical portion 22 whose diameter is smaller than that of first cylindrical portion 18. A lower end 42 of end of concentrator 30 rests on a relatively thin portion 44 (hereinafter an “acoustic port”) of housing 12, through which ultrasonic energy is coupled to the interior of the housing. Alternatively, no concentration of the ultrasonic energy is made. Optionally, the transducer is placed directly on acoustic port 44 of the housing. However, the use of concentrator 30 possibly allows for greater concentration of the energy and larger amounts of cavitation for the same input energy.

In an exemplary embodiment of the invention, acoustic port 44 serves as a barrier, for example, to allow the housing to be sterilized or disposed of without the need to sterilize concentrator 30.

In an exemplary embodiment of the invention, acoustic port 44 has acoustic properties similar to that of the liquid. In an exemplary embodiment of the invention, prevents the formation of excessive cavitation on an interface between the liquid and the transducer. While exact acoustic matching may not be possible, the closer the match of acoustic properties, the lower the amount of excess cavitation. In an embodiment of the invention, polyurethane, with acoustical properties close to those of water is used for acoustic port 44. Non-limiting suitable types of polyurethane are RTV 664 of GE Silicones (ISA) and RTV 262 of Polymer Gvulot (Israel). Other types of polyurethane may be used for the rest of the housing.

Housing 12 defines a space 24 therewithin and portion 30 of the ultrasonic device is positioned relative to housing 12 such that the end portion 32 of second cylindrical portion 22 is disposed within space 24, proximal to the wound surface 17. Thus, when device 10 or device 50 is placed over a wound, the ultrasonic waves are directed toward the wound for the purpose of cleansing.

Arms 40 are optionally used to support transducer 2.

As shown, FIG. 2 shows that portion 22 (and acoustic port 44) protrudes into the space 24. FIG. 3 shows a space in which acoustic port 44 forms part of a flat wall. The embodiment of. FIG. 1 has the advantage that the source of energy is closer to the wound than in FIG. 2. However, the embodiment of FIG. 3 is also usable.

Preferably the frequency of the transducer is not more than 80 kHz since the threshold for cavitation occurs at lower energies-for lower frequencies. However, frequencies as low as 20 kHz can be used in some embodiments of the invention. However, such low frequencies are generally not used, since they are within the range of hearing of humans and the high power used can cause problems with the patient's hearing. Thus, generally, frequencies greater than 30 kHz are used. In addition, the distance from portion 22 to the wound surface is less than one-half wavelength of the acoustic energy, preferably less than ¼ wavelength, so that the energy field is substantially uniform within the cavity. There are several reasons for keeping the cavity small. One of these is to prevent the generation of variations of power in the volume. Since the maximum acoustic energy is at the transducer, variations in energy would result in lower than optimal energy at the surface of the skin. Another, less important reason (since the cavity is generally small) is that the cavitation that would take place in the large cavity would require excessive power inputs to assure cavitation at the skin surface.

Device 10 is optionally associated with a cleansing means 33 including an inlet canal. 34, disposed within housing 12, via which a liquid is be introduced onto the surface 16 of the wound within the space 24 in order to facilitate cleansing of the wound. There is also provided an outlet canal 36, also disposed within housing 12, via which liquid containing debris, such as dirt and necrotic tissue, may be removed from the area of the wound. Optionally outlet canal 36 is disposed at the apex of space 24, for reasons that will be discussed below. Inlet canal 34 may be connected to any suitable source of cleansing liquid and outlet canal 36 may be connected to any suitable liquid removal unit (which may be as simple as a drain). Inlet canal 34 and outlet canal 36 may pass through a circular plate 38 disposed within device 10. Alternatively, if desired, the housing 12 may be provided with apertures (not shown) such that inlet canal 34 and outlet canal 36 may pass therethrough and may be fastened to portion 22 of the ultrasonic device by any suitable means. Any other suitable means for providing for the introduction and removal of cleansing fluid maybe provided.

It should be noted that sealing edge 14 optionally provides a seal between housing 12 and the surface 16 of the wound, so that cleansing liquid introduced onto the wound surface 17 does not leak onto the patient. As shown in FIG. 2, edge 14 bends outward from housing 12, which is particularly suitable where, during operation of the device 10, it is desirable for the pressure within housing 12 to be less than that outside housing 12. In this case, greater pressure outside housing 12 will apply pressure on the outside thereof, such that the outwardly protruding flange 14 will maintain a tight seal between housing 12 and the wound surface 16. In FIG. 3, the sealing edge bends inward, for use, for example, where the pressure within housing 12 to be higher than that outside housing 12

During operation of device 10, housing 12 is positioned over the surface 16 of a wound and is held in position such that a seal is formed between the surface of the wound and flange 14. Cleansing liquid is introduced into space 24 via inlet canal 34 so that space 24 begins to fill with liquid. As space 24 fills with liquid, air is forced out via outlet canal 36, then both liquid and the remaining air bubbles are forced out via outlet canal 36, until the entire space 24 is filled with liquid. It should be noted that the specific structure of device 10, wherein the outlet canal 36 is disposed above space 24, enables all of the air to be removed from the space 24. Once all the air has been removed from space 24, the flow of liquid may be terminated, if desired, and only the liquid present within space 24 may be used. Alternatively, the flow of liquid may be continued during operation of the ultrasonic device, such that liquid containing debris within space 24 that has been removed from the surface 16 of the wound may be more quickly removed from within space 24.

The present invention thus provides an improvement over prior art devices used for the cleaning of skin, wherein high energy ultrasonic sources are employed and there may be cavitation of the liquid on or near the surface of the ultrasonic transducer, possibly due to the presence of air in the cavity of the devices. In these prior art devices, there is no cavitation of the liquid on the surface of the skin, due to the presence of cavitation on the liquid surface and this results in a loss of energy transmitted to the area to be cleaned. It should be noted that in such prior art devices, if any cavitation occurred, it would be considered undesirable as it blocks ultrasonic energy from reaching the wound. Power would be reduced to prevent the cavitation.

In order to maximize the cleaning ability of the ultrasonic device of some embodiments of the present invention, cavitation of the liquid occurs on the surface of the wound (and is not suppressed by a free air surface); due to complete removal of all air from the space 24. The phenomenon of cavitation triggers a mechanical action that removes unwanted material, such as dirt and dead cells, at a molecular level, from the surface 16 of the damaged tissue. In this manner, debris may be more easily removed from the surface 16 of the wound than with other prior art devices. Alternatively or additionally, cavitation on the wound is enhanced by acoustic port 44 preventing excess cavitation at concentrator 30.

As seen in FIGS. 2 and 3 inlet 34 and outlet 36 are situated on opposite sides of wound 17. This aids in the removal of debris loosened by the ultrasound and the cavitation.

It should be noted that FIGS. 2 and 3 differ for example in two respects, namely the shape of the sealing edge and the internal shape of the space. It should be understood that different combinations of these elements are also possible.

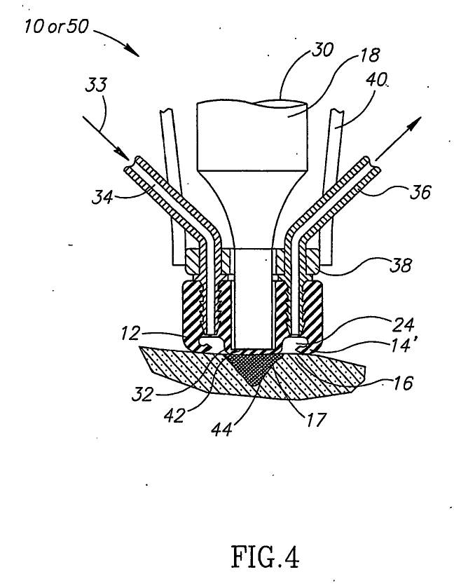

As shown in FIG. 4, if desired, pressure may be applied to housing 12, e.g., by applying pressure to the ultrasonic device, thus bending seal 14/14′ with respect to housing 12. In this manner, acoustic port 44 of device 10/50 is moved into closer or direct contact with wound surface 16, so as to enable the ultrasonic waves to be transmitted directly onto wound surface 17 and to concentrate the energy (and cavitation) in the region of the wound. In some applications of the device, acoustic port 44 is made to touch the skin, since in some medical treatments direct coupling of the ultrasound into the wound is considered desirable. Of course, such movement of the upper surface of the cavity could be performed, albeit less effectively with the apparatus of FIG. 3.

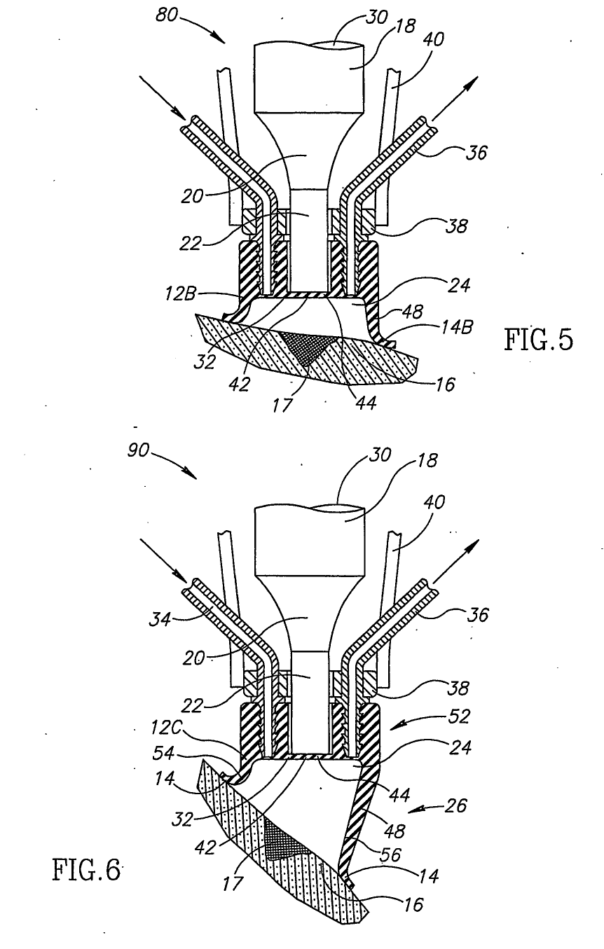

FIG. 5 shows an ultrasonic device 80 according to another embodiment of the invention. Device 80 includes a housing 12B, terminating in an elliptically shaped edge 14B, such that a longer portion 48 of housing 12B on the side of the outlet canal 36 tapers to shorter portion 46 of housing 12B on the side of the inlet canal 34. This particular structure of housing 12B enables portion 30 of the ultrasonic device to be held horizontally during operation of the device 80 on a wound whose surface is presented at a slight angle, i.e., near horizontally.

FIG. 6 shows an ultrasonic device 90 according to another embodiment of the invention, wherein device 90 is provided with a housing 12C. Housing 12C includes a first portion 26 configured such that a longer portion 56 thereof, on the side of the outlet canal 36 tapers to a shorter portion 54, on the side of the inlet canal 34. First portion 26 includes a circular flange 14 at one end thereof, to be positioned on the surface 16 of a wound. At the other end of first portion 26 and at an angle thereto, a second, cylindrical portion 52 of housing 12C is disposed circumferentially with respect to ultrasonic portion 22. This particular structure of housing 12C enables portion 30 of the ultrasonic device to be held horizontally during operation of the device 90 on a wound whose surface is presented at a large angle, i.e., not near horizontally.

It is noted that some of the above described embodiments may describe a best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. When used in the following claims, the terms “comprise”, “include”, “have” and their conjugates mean “including but not limited to”.

It will be appreciated by persons skilled in the art that the scope of the present invention is not limited by what has been particularly shown and described above. Rather, the scope of the invention is limited solely by the claims, which follow.

1. Apparatus for the ultrasonic treatment of tissue, including:
a housing having a space therewithin and an opening adapted for placement against the tissue, the housing being adapted for introducing liquid therein such that when so placed, the space is filled with liquid;
an ultrasonic power source that introduces ultrasonic vibrations toward the tissue, said vibrations having a frequency and power level sufficient to produce cavitations of the liquid at or near the surface of the tissue;
an elastic interface capable of transferring acoustic energy, separating the ultrasonic power source from the liquid.

2. Apparatus according to claim 1, wherein the opening comprises a sealing element that provides a scat at the tissue.

3. Apparatus according to claim 2, wherein the seal includes a flexible element.

4. Apparatus according to claim 2, wherein the seal includes an outwardly protruding portion that is placed to contact the tissue surface.

5. Apparatus according to claim 3, wherein the seal includes an outwardly protruding portion that is placed to contact the tissue surface.

6. Apparatus according to claim 2, wherein the seal includes an inwardly protruding portion that is placed to contact the tissue surface.

7. Apparatus according to claim 3, wherein the seal includes an inwardly protruding portion that is placed to contact the tissue surface.

8-27. (canceled)

28. A method for treating tissue, including:
providing a liquid in contact with a surface of the tissue;
producing a fluid current moving through the liquid, the current allowing for the removal of debris from the tissue surface; and
causing ultrasonic vibrations in the liquid to an extent that cavitations are caused at least at or near the surface of the tissue.

29. A method according to claim 28, wherein the frequency of the ultrasonic vibrations is not more than 80 kHz.

30-32. (canceled)

33. A method of applying ultrasound to a surface of a patient, comprising:
providing a housing having an opening at one portion thereof and having a source of acoustic energy coupled to a portion of an inner surface thereof, the housing including an interface capable of transferring the acoustic energy;
placing the opening at the patient surface, to form a substantially closed volume in the housing;
filling the volume with liquid, so that all air is removed therefrom; and
activating the source of acoustic energy to introduce vibrations towards the patient surface,
wherein the interface blocks the liquid from the source of acoustic energy.

34. A method according to claim 33 wherein the activation of the source causes the source to produce sufficient energy to cause cavitations in the liquid.

35. An apparatus according to claim 1, wherein the source of acoustic energy includes ultrasonic energy concentrator.

36. An apparatus according to claim 1, wherein the interface is part of the housing.

37. An apparatus according to claim 1, wherein the interface comprises an elastic barrier.

38. An apparatus according to claim 37, wherein the elastic barrier comprises polyurethane.

39. An apparatus according to claim 1, wherein at least a portion of the interface acoustically matches the liquid to an extent that prevents cavitation at the interface between the liquid and the interface.

40. An apparatus according to claim 1, wherein the housing is disposable or separately sterilizable and reusing the ultrasound power source docs not require sterilization thereof.

41. A method of applying ultrasound to a surface of two patients, comprising:
(a) providing a source of acoustic energy;
(b) providing a first housing having an opening at one portion thereof, the housing including an elastic barrier;
(c) coupling the source of acoustic energy to the elastic barrier,
(d) placing the opening at a tissue surface of a first patient, to form in the housing a substantially closed volume separated from the source of acoustic energy by the elastic barrier;
(e) filling the volume with liquid, so that all air is removed therefrom;
(f) activating thc source of acoustic energy to cause cavitations in the liquid;
(g) removing the first housing from the surface of the first patient;
(h) utilizing a second housing to replace the first housing and repeating (b) to (f) with the second housing and the second patient utilizing the same source of acoustic energy of (a).

42. A method according to claim 43, wherein the second housing is the first housing after sterilization.

43. A method for treating tissue, including:
providing a liquid in contact with a surface of the tissue;
providing a source of acoustic energy separated from the liquid by an interface formed of an elastomeric material; and
injecting ultrasonic vibrations from the source into the liquid to cause cavitations in the liquid, at least at or near the surface of the tissue.

44. Apparatus for the ultrasonic treatment of tissue, including:
a housing having a space therewithin, and an opening adapted for placement against the tissue to create a space between the housing and the tissue, the housing having an inlet canal for inletting liquid to the space, and a seperate outlet canal, for letting the liquid out of the space, wherein the inlet canal and the outlet canal are arranged to allow continuous flow of liquid through the space; and
an ultrasonic power source that introduces ultrasonic vibrations toward the tissue, said vibrations having a frequency and power level sufficient to produce cavitation of the liquid at or near the surface of the tissue.
